(19) United States
(12) Patent Application Publication
(10) Pub. No.: US 2007/0122488 A1
(43) Pub. Date: May 31, 2007

(54) Multi-functional microcapsules and method and device for manufacturing same

(76) Inventors: Erich Windhab, Hemishofen (CH); Michael Zimmermann, Rapperswil (CH)

Correspondence Address: WINSTON & STRAWN LLP;PATENT DEPARTMENT, 1700 K STREET, N.W., WASHINGTON, DC, 20006, US

(21) Appl. No.: 11/510,869

(22) Filed: Aug. 24, 2006

Related U.S. Application Data
(63) Continuation of application No. PCT/EP04/01840, filed on Feb. 25, 2004.
(63) Continuation of application No. PCT/EP04/01860, filed on Feb. 25, 2004.

(51) Int. Cl. A61K 9/50; B29C 39/10

(52) U.S. Cl. 424/490; 264/4

(57) ABSTRACT

Multimicrocapsules that include encapsulated components of at least one nutritionally, pharmacologically, or medically active functional substance, wherein, within an encapsulation of one or more layers of a first encapsulating material around a common capsule matrix, there are provided a first active functional component in an amount sufficient to provide functional benefits after being ingested by a subject and a component preserving or function enhancing substance present in an amount to preserve and enhance the functional benefits of the active functional component. The encapsulation delays release of the active functional component when the microcapsules are ingested by the subject and provides a synchronous or tuned release of the active functional component and component preserving or function enhancing substance in the intestine of the subject. Also, a process and apparatus for forming such microcapsules.

### CROSS-REFERENCE TO RELATED APPLICATIONS

This application is a continuation-in-part of International Applications PCT/EP2004/001840 and PCT/EP2004/001860, each filed Feb. 25, 2004, and the entire content of each which is expressly incorporated herein by reference thereto.

### BACKGROUND

The invention relates to multimicrocapsules encapsulating several functional substances or substance components that are effective in the sense of their pharmaceutical, medicinal or nutritional properties. The invention also relates to a method and device for producing such multimicrocapsules.

In the fields of pharmacy and medicine, cosmetics and nutrition, functional substances or substance components play an increasing role from the standpoint of their defined release, e.g., in the human gastrointestinal tract in a form or with release kinetics that ensure the best possible uptake by the body or the best possible bioavailability and efficacy. Such functional substances generally include pharmaceutical active ingredients or medications in pharmacy or medicine; dermatologic ingredients in cosmetics; and nutritional components in foods. In storage of reactive functional substances and substance components, there are often interactions with the ambient substance matrix and, associated with that, definite losses with regard to the efficacy of the functional substances/substance components occur.

In other applications, the efficacy of functional substances or components is also greatly impaired by their interaction with other substances that are present in the human digestive tract. Such substances having adverse interactions in the digestive tract may originate from foods as well as being constituents of the digestive system. For example, iron preparations administered to eliminate the signs of iron deficiency are oxidized under the boundary conditions prevailing in the human stomach (low pH) and because of the oxidative effects in interaction with phyto acids from edible plants or milk products, for example, thereby impairing their absorbability in the small intestine and thus limiting their bioavailability.

For an exemplary discussion of a possible synergistic approach to elimination of symptoms of a nutritional deficiency, reference is made to interaction effects with deficiencies of iron, iodine and vitamin A. Iron deficiency anemia, vitamin A deficiency and enlarged thyroid (goiter) caused by an iodine deficiency affect more than one-third of the world's population. These deficiencies often occur concurrently, e.g., in regions of western and northern Africa, where approximately one-third of school-age children suffer from concurrent deficiencies of iron, iodine and vitamin A. These three nutritional components are of enormous importance for the human metabolism. Iron and vitamin A deficiencies interfere with thyroid metabolism of iodine and reduce the efficacy of iodized salt. A vitamin A deficiency also impairs iron transport in the body and exacerbates iron deficiency anemia. This makes a combined approach to combat iron deficiency, iodine deficiency and vitamin A deficiency appear especially suitable as an example of an effective and synergistic strategy for fortification of foods.

The following main questions arise with regard to administration of functional substance components or active ingredients:

how can these substances be transported to the site of the desired release and absorption (e.g., small intestine) without losing quantities and effects during storage and in preparation for administration (e.g., integration into food dishes)?

how can the absorption conditions at the site of release be optimized to achieve maximum bioavailability?

These questions are now answered by the present invention.

### SUMMARY OF THE INVENTION

The invention relates to multimicrocapsules comprising encapsulated components of at least one nutritionally, pharmacologically, or medically active functional substance, wherein, within an encapsulation comprising one or more layers of a first encapsulating material around a common capsule matrix, there are provided a first active functional component in an amount sufficient to provide functional benefits after being ingested by a subject and a component preserving or function enhancing substance present in an amount to preserve the functional benefits of the active functional component. The encapsulation delays release of the active functional component when the microcapsules are ingested by the subject and provides a synchronous release of the active functional component and component preserving or function enhancing substance in the intestine of the subject.

The first active functional component is typically one that is deteriorated by oxidation, so that the component preserving substance is an antioxidant and the first active functional component is associated with the component preserving substance in the encapsulation. Alternatively, the first active functional component and component preserving substance are separately encapsulated so that they do not come in contact with each other until after ingestion and release in the intestine of the subject.

Generally, a second active functional component that is different from the first active functional component is provided within the encapsulation in an amount sufficient to provide a different, enhanced or synergistic functional benefit after being ingested by a subject. If desired, the second active functional component is separately encapsulated so that it does not come in contact with the first functional active agent prior to release in the subject.

Advantageously, if the embedded functional component is hydrophilic the common capsule matrix comprises a hydrophobic fat or wax material that suppresses diffusion-based exchange of the active functional component and the component preserving or function enhancing material during storage of the microcapsules prior to ingestion, or if the embedded functional component is hydrophobic the common capsule matrix is a hydrophilic material that forms a gel to separate the first active functional compound from the component preserving or function enhancing material during storage of the microcapsules prior to ingestion.

Preferred first encapsulating materials include a lecithin compound, a sugar ester, a polyglycerol ester, or a mono- or di-glyceride compound present in one or more layers to provide the encapsulation. Also, preferred first active functional components include a vitamin, a mineral, a metal compound, an aroma or an oxidizable drug, with preferred component preserving or function enhancing substance materials including an antioxidant, an enzyme, a buffer system, an acid, a salt or a halide. Alternatively, the first active functional compound can be an aroma-generating component, present with a separately encapsulated component that is reactable with the aroma generating component to react, generate and release the aroma when the components contact each other after release from the microcapsules.

Another embodiment of the invention relates to a method of providing a nutritionally, pharmacologically, or medically active functional substance to a subject, which comprises administering the microcapsules described herein to a subject in need of such treatment, wherein the microcapsules are administered to provide an amount of the active functional substance to the subject provide functional benefits thereto. Typically, the microcapsules are provided in a foodstuff or beverage and the administration of the microcapsules is provided by the consumption of the foodstuff or beverage by the subject.

Particular treatments include treating a vitamin, mineral or metal deficiency in a subject by administering to a subject in need of such treatment microcapsules containing as the first active functional component a vitamin, a mineral, or a metal compound, and delivering an oxidizable drug to the intestine of the subject by administering to a subject in need of such treatment microcapsules containing as the first active functional component, an oxidizable drug and an antioxidant as the component preserving or function enhancing substance, such that the first active functional component and antioxidant are preserved by the encapsulation until released in the intestine of the subject and after release the component preserving or function enhancing substance prepares the environment for maximum reaction or uptake or minimized loss of the first active functional component. By these methods, iron or vitamin deficiencies are easily treated with unexpectedly improved results.

Another embodiment of the invention relates to a process for producing microcapsules containing at least one nutritionally, pharmacologically, or medically active functional substance. This method includes the step of incorporating within an encapsulation comprising one or more layers of a first encapsulating material around a common capsule matrix, a first active functional component in an amount sufficient to provide functional benefits after being ingested by a subject and a component preserving or function enhancing substance present in an amount to preserve or enhance the functional benefits of the active functional component, wherein the encapsulation delays release of the active functional component when the microcapsules are ingested by the subject and provides a synchronous release of the active functional component and component preserving or function enhancing substance in the intestine of the subject.

Generally, at least two functional substances are provided in the encapsulation, with each substance optionally being encapsulated separately. The process includes premixing of the functional substance components and then milling or dispersing them in fluid matrix phases to produce the subcapsule-suspension or emulsion with a solid functional substance. These suspensions or emulsions are sprayed into a cold spraying chamber or spray drying tower to form liquid suspension or emulsion drops in which the liquid phase is then solidified by freezing or gelling (e.g., by cold spraying), glassifying or solidifying (e.g., cold spraying or spray drying) or concentrating and gelling (e.g., by spray drying). The gelling and glassifying or solidifying steps can be adjusted by adding solutes to the fluid matrix. Such solutes include phases like sugars, salt, oligi- or poly-saccharides and proteins. The encapsulating layers may be provided by surfactant substances that are as well added to the fluid matrix phases and which due to their amphiphilic nature arrange at the surface of the sprayed capsule drops or at inner interfaces between solid and liquid phases of suspensions or the liquid/liquid phases of emulsions. In addition, the surface of the subcapsules may be spray coated by the cold spray or spray drying methods whereby a coating fluid is sprayed onto the subcapsules formed and partially solidified in a cold gas or hot gas drying atmosphere forming a layer or layers on the subcapsule surface which then are solidified in crystalline or vitreous form by withdrawing heat or by drying.

The sub-capsules received represent the simplest case of single functional component containing capsules for which a second preserving and/or function enhancing substance could be part of the matrix phase or one of the layers formed. In the general case of the invention different sub-capsules containing active functional components 1-n or containing protective or function enhancing substances 1 to n are separately produced allowing to adjust the surrounding matrix and layer materials to the specific protection and release requirements of the embedded active functional component.

In order to produce the capsules (i.e., the main capsules) containing two or more sub-capsules with active functional components or preserving or function enhancing substances, respective sub-capsules are mixed (A), redispersed in the liquid main capsule matrix phase thus forming a sub-capsule suspension (B) and then again sprayed within a cold spraying or spray drying device to form sub-capsule suspension drops which are then solidified and eventually spray coated forming the main capsules containing sub-capsules as composed.

The invention also relates to a device for producing microcapsules containing at least one nutritionally, pharmacologically, or medically active functional substance, comprising static or dynamic mixer or stirrer tools for premixing the functional substance(s) in a fluid matrix phase, a first dispersing device for defined fine dispersion or size reduction of the functional substance(s); a drying device for producing subcapsules and a second dispersing device for producing main capsules which contain multiple subcapsules with the overall device components arranged one after the other in sequential order and are to be operated in this order either batchwise or continuously. Advantageously, the first dispersing device comprises a rotor-stator dispersing apparatus, a high pressure homogenizer, an ultrasound dispersing device, a membrane dispersing apparatus, or a microfluidic dispersing channel, the drying device comprises a cold spraying chamber or a spray drying tower, and the second dispersing device comprises a mixing or stirring vessel for sub-capsule suspension mixing or dispersing and a second cold spraying chamber or second spray drying tower.

### BRIEF DESCRIPTION OF THE DRAWING FIGURES

Other features and advantages are derived from the following detailed description which identifies preferred embodiments of the invention and examples, partially in schematic diagrams, in which:

FIG. 1 shows a multimicrocapsule morphology in a roughly schematic diagram;

FIG. 2 is a micrograph of multimicrocapsules in foods with a fat matrix in the digestive tract,

FIG. 3 depicts crystalline functional substance phases with an amorphous layer surrounding them and an additional external amorphous boundary layer;

FIG. 4 presents a process diagram to illustrate preferred process steps; and

FIG. 5 illustrates a preferred device according to the present invention.

### DETAILED DESCRIPTION OF THE PREFERRED EMBODIMENTS

The invention relates to multi-microcapsules containing two or more functional components adjusted in type and quantity to support a synergistic interaction, the components contained within a common main capsule with tailored multiphase morphology determining diffusion and reaction kinetics of the functional components, and being structured such that under ambient storage conditions no functional component interaction takes place, however under application conditions optimum control of the reaction between the functional components in the environment where they are released, is received.

In these multi-microcapsules, the multiphase-morphology of the main capsule is a main capsule matrix phase (i) with embedded two or more types of sub-capsules (ii) consisting of similar or different sub-capsule matrix phases and with functional components (iii) embedded in these sub-capsule matrix phases. The main capsule, sub-capsules and functional components are preferably covered by interfacial films of interfacially active molecules. The matrix phases and interfacial layers generally are made of materials which on one hand protect the encapsulated functional components from too early release and on the other hand disintegrate when the desired position for release is reached.

When hydrophobic fats or waxes are used as the materials for the matrix phases or the interfacial layers, the functional components are protected within a watery environment yet still can be disintegrated by enzymes such as lipases acting within certain zones of the capsule track to the desired position of release. When hydrophilic watery systems in a glassy or gel state are used as materials for the matrix phases or interfacial layers, the functional components are protected within a hydrophobic environment yet still can be disintegrated by enzymes such as cellulases or proteases acting within certain zones of the capsule track to the desired position of release.

Preferably, several similar or different interfacial layer coatings are deposited on the main capsule, sub-capsule or functional components in such an order that protection of the functional components is reached or so that controlled disintegration or dissolution of the respective layers is reached for the altering surrounding conditions along the multi-microcapsule track to its position of desired release and reaction.

When a fat-based main capsule matrix phase is used, it is preferred for that phase to have a melting temperature above that of human body temperature, providing on one hand sufficient mechanical and chemical stability when passing the mouth, throat, larynx and stomach to protect the embedded sub-capsules and contained functional components and on the other hand gets disintegrated by lipases from the gall juice when entering into the small intestine, consequently releasing the sub-capsules. A similar result can be achieved with the use of a watery polysaccharide matrix phase with gel structure which when passing the small intestine gets that far dissolved or disintegrated by water soluble enzymes contained in the gastric juice eventually also originating from another sub-capsule or even from a matrix or layer phase in which it has been included before, that the functional components embedded in the sub-capsules are released and get in contact with the small intestine mucosa.

The multi-microcapsules according to the invention can have different component constructions. One arrangement has the combination of a function carrying component and an antioxidant which are contained together, with the antioxidant protecting the function carrying component from oxidation. Alternatively, the function carrying component and activation component are separately contained, so that the activation component is adjusted as function of activating the function carrying component. Another variation is that two or more different function carrying components are provided with one or more of these components selected and adjusted to activate a synergistic interaction with another of these components to enhance the function of that component or to create a new function. Yet another arrangement is that two different functional components are provided which when released react to form a reaction product that provides a different function than that of the components. The release and reaction kinetics are adjusted by the characteristic diameter and thickness dimensions of the main capsule, any sub-capsules, and the selection of the functional components and the interfacial layers, as well as their relative sizes, amounts or thicknesses.

Typically, these multi-microcapsules have typical diameters of the main capsule between some microns and some millimeters, preferably between 50 and 500 microns, while the typical diameters of the sub capsules range between 1 and 200 microns, and preferably between 1 and 10 microns. Also, typical diameters of the functional components whether of solid particle or liquid drop nature range between some nanometers and 50 microns, and preferably between 50 and 500 nanometers. The typical thickness of the interfacial layers range between 1 nanometer and 10 microns, preferably between 10 and 100 nanometers. Furthermore, the main capsules, sub-capsules or solid particle-based functional components may have spherical to fiber-like shapes to adjust or modify the disintegration, component release and component interaction reaction kinetics of the encapsulated components. Such multi-microcapsules can contain functional components the synergistic interaction of which is of either nutritional, health-related, aroma-/flavor-related, texture/rheology-related or stability related.

A preferred embodiment of the present invention relates to a multimicrocapsule for incorporating multiple pharmacologically, medically or nutritionally active functional substances or substance components therein in such a way as to suppress interactions of the encapsulated substances or substance components under storage conditions on the one hand, while on the other hand permitting a definitively adjustable release of the encapsulated substances or substance components with regard to the point in time of release and rate of release with administration in coordination with environmental conditions. Another goal is to combine the encapsulated substances in such a way as to achieve positive synergistic effects (e.g., mutual protective effect against oxidation, improved bioavailability) during storage, release and interaction.

Several substances/substance components having functional properties can be incorporated into a multimicrocapsule in such a way that no interaction of the encapsulated substances or substance components occurs during storage of such capsules, i.e., stability in storage is ensured.

Another important advantage is derived from the spatial proximity of the substances/substance components enclosed in a microcapsule because this leads to contact in their release from the microcapsule and to targeted synergistic interactions as a result of the respective combination.

Another advantage is the inventive adjustability of the time of release and the rate of release of encapsulated functional substances/substance components through a suitable choice of capsule materials, coordinated with the ambient conditions during use, forming enveloping layers around these functional substances as well as the layer thickness and microstructure thereof.

For example, nutritional substance components such as water-soluble iron salts (as a first substance component) may be transported by means of multimicrocapsules into the small intestine of volunteers having an iron deficiency under conditions that protect the iron salts from oxidation, then releasing these salts from the capsule in the small intestine in synchronization with an antioxidant (as a second substance component). The intense interaction of these components in the microenvironment of the capsule protects the iron salt from oxidation and significantly improves the bioavailability of iron, i.e., its absorption in the small intestine.

Thus the inventive multimicrocapsules allow the incorporation/encapsulation of multiple functional substances or substance components in one multimicrocapsule with the option of adjusting dividing layers between these substances/substance components with regard to diffusion permeability, time of release and release kinetics. This meets the prerequisite for production of a multimicrocapsule that is suitable with regard to storage stability and optimized release of multiple functional synergistically interacting substances or substance components.

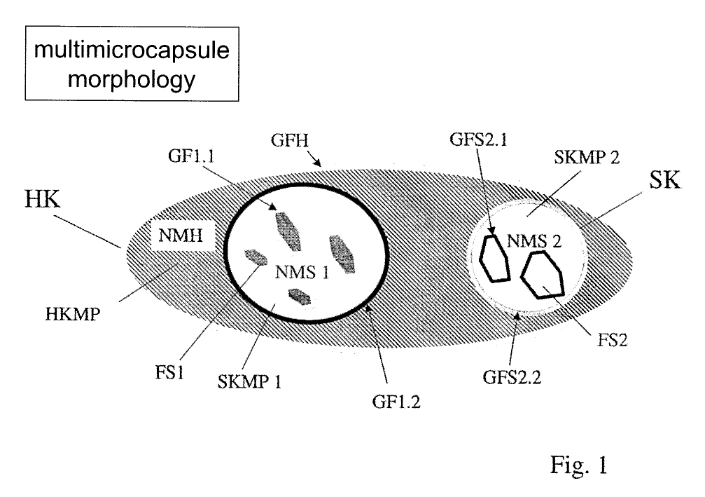

For illustration, the complex structure of a multimicrocapsule is shown in FIG. 1. According to this invention, multimicrocapsules are comprised of subcapsules (SKi) having encapsulated functional individual substance components (FSi) in which these SKi are embedded in a main capsule matrix phase (HKMP). This main capsule matrix phase is selected so as to largely suppress a diffusion-based exchange of encapsulated functional substances/substance components (FSi) under storage conditions.

According to this invention, the FSi are preferably used in solid form, usually in crystalline form. The various FSi are preferably each enclosed in a subcapsule matrix phase (SKMPi). According to this invention, These SKMPi are selected to be opposite the FSi with regard to their hydrophilic/hydrophobic property. The molecular structure of the SKMPi is preferably largely amorphous (vitreous) based on the composition and method of production, and may optionally also be adjusted to crystalline or gel form. By means of surfactant substances, additional interfacial layers (GFi) are created as diffusion barriers between SFi and SKMPi as well as between SKMPi and HKMP as needed. Finally, such an interfacial layer may also surround the main capsule (GFH).

To adjust the time of release of the SKi and their release kinetics, the following parameters of the multimicrocapsules are adapted according to this invention.

Geometric Parameters

Diameter of the FSi particles: a few nanometers to 50 micrometers

Diameter of the subcapsules (SKi): 1 to 200 micrometers

Diameter of the main capsules (HK): a few micrometer to a few millimeters

Density of the interfacial layers (GSi)

Shape of the FSi, SKi and HK (increase in interfacial area)

Structural Parameters

Vitreous or crystalline structure of the FSi, SKMPi, HKMP

Polymorphism in crystalline structures

Single-/multiphase structure

Physical Material Parameters

Phase transition temperature of the FSi, SKMPi and HKMP

Interfacial tension/contact angle between neighboring phases

Hydrophobicity/hydrophilicity of the phases

Diffusion coefficients

Water absorbing capacity (aw value, sorption isotherms)

In principle, there is the possibility of embedding multiple FSi in solid or liquid form in one subcapsule matrix phase. The main capsule matrix phase in this case corresponds to an additional coating layer. This may optionally also be omitted.

Individual encapsulation of the FSi in subcapsules and increasing the number of “enveloping layers” (GFi, SKMPi, HKMP) make it possible to improve FSi-specifically the stability properties of the capsules on the one hand under storage conditions while on the other hand these enveloping layers allow flexible FSi-specific coordination with the boundary conditions of use.

The purpose of using microcapsules is generally the transport of functional substances/substance components FSi to the preferred site for their release and the most definitive possible adjustment of their release kinetics.

An additional goal of the inventive multimicrocapsules is to release multiple FSi in immediate proximity simultaneously or in a definitive sequence and thus permit a reaction of the FSi thereby released without any significant interfering influences of substance components from the environment. The combined encapsulated FSi are coordinated according to this invention in such a way that synergistic interactions are achieved. For example, the interaction of an antioxidant (FS1) with a nutritionally or pharmacologically/medically relevant compound (FS2) that has a good solubility in an aqueous digestive medium provides oxidation protection of FS2 and thus improved absorption and bioavailability of FS2.

A plurality of “enveloping layers” around the FSi allows the implementation of specifically adapted protective functions against varying ambient conditions along the path of the multimicrocapsules to their “destination site” (or to the site of release of FSi). For example in transport in the human digestive tract, passage through the following main zones is associated with specific stresses: A. mouth/throat area (pH neutral; great mechanical stress, water-soluble enzyme activity); B. stomach (low pH; mechanical stress); C. pylorus/small intestine (neutral pH, introduction of enzymes (e.g., fat-soluble lipases bile), water-soluble enzymes in digestive juice).

In many cases, before uptake into the human gastrointestinal tract, there are additional increased requirements of mechanical stability and thermal stability (e.g., in preparing foods containing these multimicrocapsules).

For the selected example, the enveloping layers allow the adjustment of the thermal, mechanical, pH and enzymatic stability properties. Degradation of the corresponding enveloping layer may also be desired to ensure the release of the FSi at the target site. In the selected example of the human digestive tract, fat-based layers are degraded by the action of lipases and macromolecular networks in water-based layers are degraded by water-soluble enzymes (e.g., cellulases, proteases) in the pylorus or small intestine or by dissolution in the gastro-intestinal juice. Adjusting the thickness of such layers makes it possible to shift the site of release of FSi, e.g., along the small intestine or influence the FSi release kinetics.

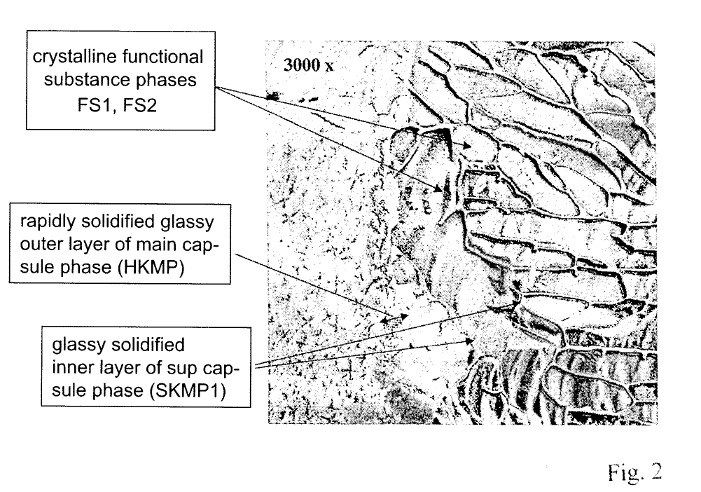

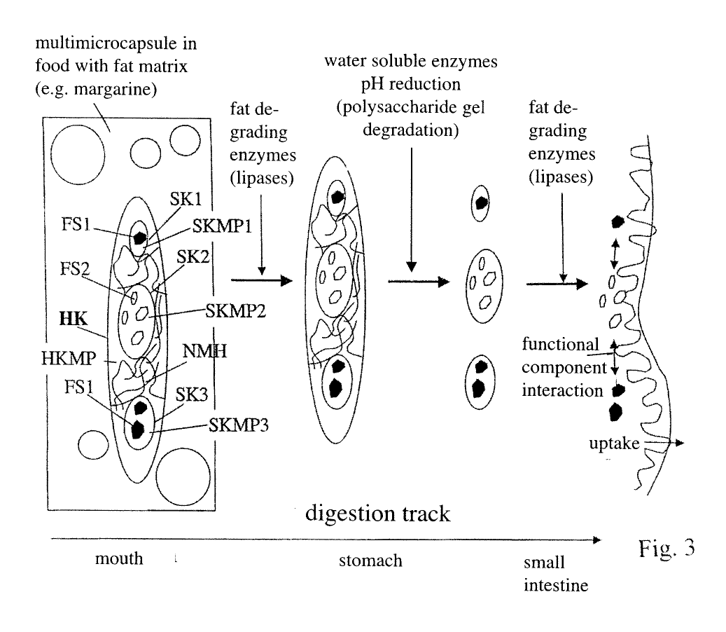

FIG. 2 shows stepwise digestion of a multimicrocapsule in the human gastrointestinal tract diagrammed schematically as an example in a photomicrograph. FIG. 3 shows the stepwise digestion of a multimicrocapsule in the human gastrointestinal tract illustrated schematically, for example.

An exemplary embodiment is presented below with a specific administration of a multimicrocapsule which contains as functional FSi in encapsulated form the substances iron pyrophosphate (functional nutritional component: iron), sodium iodate (functional nutritional component: iodine) and retinyl palmitate (functional component: vitamin A). These components are encapsulated in hydrogenated (hardened) palm kernel fat as the common main capsule matrix phase. In addition, soy lecithin was used as the surfactant substance to form interfacial layers (GSi, HGS) around the FSi (here=subcapsule matrix phase) and the capsule surface.

These iron (Fe)-iodine (I)-vitamin A multimicrocapsules (abbreviated: FeIA CAPS) with an average diameter of approx. 100 micrometers (μm) contain approx. 40% FSi (substrate) and were added to non-pulverized table salt (particle size approx. 1 to 2 mm) as a nutritional fortification component in a ratio 60 μγ vitamin A/25 μg iodine/2 mg iron/1 g NaCl (corresponding to 6:2.5:200:100,000). The iron content proved to be stable in production of FeIA CAPS. With regard to vitamin A, approx. 30 to 35% production losses occurred and with iodine production losses amounted to 15 to 20%. These losses have been compensated through corresponding overdosing to obtain the optimum concentrations in fortified NaCi batches.

Another exemplary embodiment is a phytase (enzyme)/iron (Fe) or zinc (Zn) combination encapsulated in the same manner as the previously mentioned FeIA CAPS. In general, when iron or zinc compounds or both are ingested, phytic acid in the gastrointestinal tract reacts with such compounds to cause substantial losses of the bioavailable amounts of such compounds. The phytase enzyme degrades phytic acid to reduce iron and zinc compound losses, thus making these micronutrients available.

Another component that can be encapsulated is folate or folic acid.

In these applications, a surfactant component can be added as an additional functional component which is expected to allow for improved transfer of the nutritive components through the mucous membrane into the blood circuit. Such exemplary surfactants include lecithin, soy lecithin, sugar esters, polyglycerol esters, and mono- or di-glycerides. These compounds can be added in addition to their presence as the encapsulation layers on the components or combination of components to form the microcapsules.

Yet another embodiment is the use of the microcapsules of the invention to protect and later activate easily oxidizing flavors and flavor generating components. In this embodiment, the flavor or flavor generating component comprises an acetal component and optionally an antioxidant component. Any organic acetal can be used but those having between 4 and 12 carbon atoms are desirable. The acetal component is preferably trans-2-hexanal (e.g., 98.5%, propanediol 1% and citric acid 0.5%). Any conventional pH7 buffer component is also present in the microcapsules so that it can eventually react with the acetal component.

The acetal component and antioxidant(s) are separately encapsulated as is the pH7 buffer component and included in the microcapsules. Thus, during manufacture and storage, the acetal and buffer components are separated and consequently do not interact. During consumption of the microcapsules, the barrier layers between the two components are disintegrated (either by melting, plasticizing or dissolution) and the components start to interact. Interaction means that the buffer breaks down the acetal to release the flavor. The antioxidant component supports the protection of the acetal component from oxidation during storage which would result in aroma loss.

In general, the aroma-generating component that can be encapsulated according to the invention include a component that experiences a reaction of itself or with another component to cause a change in, e.g., pH, ion concentration or temperature which in turn triggers the modification of the aroma compound to transfer it from a non-activated to an activated aroma state. The skilled artisan is well aware of these type compounds so that no further elaboration is needed here.

Yet another embodiment relates to the protection of easily oxidizing drug compounds or components. That compound or component is combined with an antioxidant component (such as Vitamin A or C). This mixture is encapsulated by one or more of lecithin, sugar esters, polyglycerol esters, or mono- or di-glycerides as previously noted.

Again, during manufacture and storage, the drug and antioxidant components are separated and consequently do not interact. During consumption and digestion, the encapsulating or barrier layers between the components are disintegrated (e.g., by melting, plasticizing or dissolution) and the components start to interact. Interaction means that the drug is released (e.g. in the intestine) and reaches the mucous of the small intestine to get transferred into the blood circuit without losses by oxidation due to the protective action of the antioxidant that is also released in the intestine also to prepare the intestinal environment accordingly. The encapsulating layers of the types described herein also are expected to improve and speed up (or catalyze) the transfer of the drug component through the intestine mucous membrane.

Additional main- and sub-capsule components can include a number of different materials or components. These can primarily be divided into two groups:

(1) Water-based hydrophilic materials: including watery solutions of sugars, oligo- or polysaccharides or proteins which are preferably applied in concentrations where they form gels (polysaccharides at a concentration of 0.1 to 1% by weight based on the aqueous phase: e.g., locus bean gum, guar gum, tara gum, gellan and pectin; proteins at a concentration of 0.5 to 20% by weight based on the aqueous phase; e.g., gelatin, whey proteins, ovalbumin, lysozyme, casein, total milk proteins, soy proteins) and rubbery or glassy structures (sugars, oligsaccharides at concentrations of 10 to 85% by weight based on the aqueous phase).

(2) Hydrophobic materials: including fats or fat fractions (e.g., cocoa butter, coconut fat, palm fat, milk fat fractions, vegetable oils) and waxes (e.g.: Beeswax, carnauba wax, candelilla wax, soy wax, or mineral waxes).

Within the microcapsules, solidified fats with melting temperatures above the human body temperature (37 C) are preferred. Within the sub-capsules also liquid fats (e.g. vegetable oils) can be provided for suspension of solid functional components or for emulsification of liquid functional components.

Lecithin, sugar esters, polyglycerol esters, and mono- or di-glycerides can be used as the encapsulating layers, for either or both components or to form the final microcapsules themselves. As noted, each component can be individually encapsulated, or an active functional component and a protective component, such as an antioxidant, can be encapsulated together. These encapsulating or coating layers can cover the sub-capsules or the main capsule as single or multiple layers.

Additional surfactants that can be used as the coating or encapsulating layers include acid esters of mono- and diglycerides, di-acetyltartaric esters of monoglycerides, sucrose esters, sorbitan esters, polysorbates, propylene glycol fatty acid esters, stearoyl-2-lactylates, oleoyl lactylates or ammonium phosphatides.

A typical number of layers in the encapsulation is 1 to 2 or 3. In principle the main capsule and sub-capsule matrix phases form a kind of “thick layer” around the functional components. More layers allow the more specific protection steps adapted to changing conditions along the capsule track and provide greater or longer protection on the track through the gastro intestine. These interfacial layers have a total thickness of between 5 and 50 nanometers (for molecular monolayers) and up to several microns (for multilayers). If layers without molecular orientation are deposited, e.g. by spraying, they may have a thickness as great as 10 to 500 micrometers.

In addition to the surfactants mentioned herein, these interfacial layers can be formed by other amphiphilic molecules which have a hydrophobic part and a hydrophilic part. These parts orient between oil or fat and water interfaces thus forming layers with specific morphologies. Surfactants preferred for the multi-microcapsules are lecithin, sugar esters, polyglycerol esters, mono- or di-glycerides and proteins. Coating layers are principally applied by spraying them onto the solidified capsule or matrix phases. In order to form glassy layers these solutions are rapidly cooled, frozen or dried.

The microcapsules may include subcapsules if desired. The preferred relationship of sub-capsules is the adjustability of the different functional components to achieve the best protection of the active functional component (for different needs e.g. with respect to excluding oxygen or other molecules that can cause interaction during storage), as well as to prevent transfer and reaction conditions between them. If one component must be released faster or at a different rate than another or than a reacting component, sub-capsules can be adapted in size, matrix phase type and aggregate state, covering layer type, thickness, or structure state in order to provide release of one component before the other to achieve optimum interaction/reaction conditions between the different components.

When two components are to be equally treated, they can just be embedded in a common main capsule matrix phase and then be encapsulated together. If only one functional component needs a specific protection or release characteristics only this component will be embedded in a sub-capsule with the other components being contained in more simple sub-capsule or they can even be unencapsulated but present within the main-capsule. The main capsule guarantees that a well-defined fraction and composition of all reaction partners or components are provided for transport to a distinct place for release/reaction. It also assures that the reaction partners are released and are able to find each other easily within the micro environment around themselves for the desired reaction. Otherwise, the reaction required would be non efficient or incomplete.

In each of these embodiments, the microcapsules are added to a foodstuff or beverage to facilitate ingestion by a subject. The microcapsules can be added to any type of food product, either by themselves or with conventional food additives such as a seasoning. The microcapsules of the invention can also be added to any beverage or beverage composition for administration to a subject.

In particular, table salt (NaCl) is an ideal vehicle for nutritional enrichment of foods and therefore is also suitable for experiments in “three-component enrichment” with iron, iodine and vitamin A. In the past such enrichment has not been successful because the water-soluble iron compounds would result in unacceptable changes in color on exposure to moisture from salt or ambient air and impurities in table salt and iodine and vitamin A in enriched table salt are oxidized in the presence of moisture and oxygen and therefore are lost.

The table salt enriched with FeIA-CAPS has been tested in Africa (Morocco). Storage stability tests were performed at 0, 2, 4 and 6 months after combining the multimicrocapsules with the table salt. The enriched table salt was packaged in transparent 2 kg polyethylene bags and exposed to the influence of indirect daylight in rooms. Table 1 shows the average concentrations of vitamin A, iodine and iron of twelve 50 g salt samples tested at each storage time.

TABLE 1
Stability of Vitamin A, iodine and iron content of
“triple-fortified” table salt (averages).

| Month of salt storage | n | Vitamin A (μg) | Iodine (μg) | Iron (mg) |
|---|---|---|---|---|
| 0 | 12 | 69.7 | 26.5 | 1.9 |
| 2 | 12 | 74.5 | 24.7 | 2.1 |
| 4 | 12 | 69.4 | 22.2 | 2.0 |
| 6 | 12 | 61.2 | 22.6 | 1.9 |

The values listed in Table 1 emphasize the very good storage stability of the nutritional substance components encapsulated in FeIA CAPS. In comparison with previous experiments in an attempt to achieve a corresponding enrichment of salt, these results obtained with the multimicrocapsules described here show a definite improvement.

Without the corresponding encapsulation, vitamin A losses of 80 to 90% were observed in one month, and iodine losses of approx. 40 to 50% were observed in six months.

For these samples of table salt enriched with FeIA CAPS, tests were also performed to determine the absorption, bioavailability and efficacy in a random double-blind test series with 184 Moroccan school children between the ages of 6 and 15 who had a high incidence of anemia, enlarged thyroid and vitamin A deficiency. Families participating in the study were divided into two random group. One group received only NaCl enriched with 30 μg iodine/g salt. The other group received the triple-fortified salt in the form of FeIA CAPS (triple-fortified salt TFS).

Each family received 2 kg salt at the start of each month for five months to cover all household needs. After five months, the same blood and urine tests were repeated on the 184 children included in the study. Table 2 shows the comparative results obtained.

TABLE 2
Results of the efficacy study for comparison of triple-fortified salt
(TFS; FeIA-CAPS) with iodized salt (¹averages; ²percentage values).

| | Iron status | | | Vitamin A status | | |
|---|---|---|---|---|---|---|
| | | | % of | | % of | |
| | | | children | | children | Iodine status |
| | | Serum | with iron | Serum | with | Urinary |
| | Hemoglobin | ferritin | deficiency | retinol | vitamin A | iodine |
| Group and time | (g/L)¹ | (μg/L)¹ | anemia² | (μmol/L)¹ | deficiency² | (μg/L)¹ |
| Iodized salt | | | | | | |
| Starting value | 10.9 | 12 | 34 | 0.99 | 5 | 12 |
| 5 months | 10.8 | 13 | 32 | 0.98 | 7 | 76 |
| Triple-fortified salt | | | | | | |
| Starting value | 10.8 | 14 | 35 | 0.95 | 12 | 11 |
| 5 months | 12.8 | 45 | 21 | 1.14 | 0 | 72 |

As the results presented in Table 2 show, vitamin A, iodine and iron were bioavailable in the triple-fortified TFS salt samples (FeIA CAPS) and had a very good efficacy in the school children included in the study. Both the iron and vitamin A status improved significantly in the TFS group but not in the comparative group. The iodine status of both comparative groups showed a comparable trend, as expected.

This example emphasizes the efficacy of multimicrocapsule with regard to achieving good storage stabilities that could not be achieved in the past as well as improved functionality in the sense of bioavailability of the nutritional substance components tested.

Another system that can be utilized is the following:

Phytase (enzyme)/iron/zinc combination reducing the iron and zinc losses caused by phytate (e.g. from plant food) interactions in the intestine, thus improving iron and zinc availability.

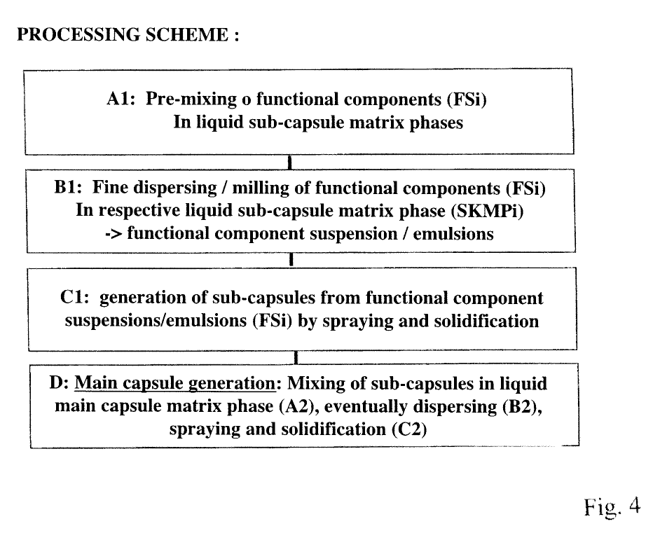

The multimicrocapsules of the invention can be produced in four main steps according to the process diagram shown in FIG. 4 and described in detail below.

Step 1: PREMIXING—First the FSi are premixed with the corresponding fluid subcapsule matrix phases (SKMPi). Mixing/stirring systems consisting of heated mixing containers and stirrers are preferably used to do so.

Step 2: DISPERSING—Wet milling in the case of solid-based FSi/droplet dispersing with fluid FSi in a non-miscible fluid phase which contains the subcapsule matrix phase SKMPi or at least one of their main components, and preferably, according to this invention, surfactant components GFi to form interfacial layers (GFSi) between functional substance components FSi and the subcapsule matrix phase SKMPi plus optionally network-forming macromolecules (NMSi) which in case of watery phases cause the SKMPi to undergo a gelatinous crosslinking process.

Wet milling is preferably performed in a ball mill with grinding bodies coordinated in size with the milling fineness to be achieved, preferably made of a ceramic material. As an alternative, roll mills or hammer mills may also be used.

The droplets are preferably dispersed in rotor/stator droplet dispersing devices (e.g., colloid mill or crown gear disperser) or high-pressure homogenizers with adjustment of a very narrow droplet size distribution in capillary jet extensional flow dispersion channel (K-JetDSDK), as is preferred according to this invention.

To adjust the narrowest possible solid particle/droplet size distributions in the FSi, according to this invention a classifying step (=separation according to particle size/droplet size) is preferably performed (substep to step 2) by means of wet screening (i) or sedimentation separation in a centrifugal field (ii) downstream from the milling/dispersing step and the particles/droplets that are too large are recycled back to the milling/dispersing chamber.

Step 3: PRODUCING SUBCAPSULES—The subcapsules (SKi) are preferably produced according to this invention by spraying a suspension/emulsion consisting of the finely ground/finely dispersed, optionally classified and GFi-enclosed FSi particles/droplets suspended/emulsified in the SKMPi into a cold space (cold spray tower). The temperature of the space is preferably selected according to this invention to be <20-50° C. below the phase transition temperature or gelation temperature of the SKMPi with network-forming macromolecular substances (NMSi) optionally contained therein.

The temperature of the spray space is selected so that a vitreous/amorphous (1), crystalline (2) or gelatinous (3) solidification of the SKMPi is achieved in a targeted manner.

This vitreous/amorphous state (1) is achieved with water-based and fat-/wax-based SKMPi and with high temperature gradients; state (2) is also achieved with water-based and fat-/wax-based SKMPi under temperature gradients that are not as great; state (3) refers only to water-based SKMPi with comparatively moderate cooling temperature gradients or even under heating conditions. If multimicrocapsules are to be stable in storage under room temperature conditions, then water-based SKMPi should be solidified only according to mechanism (3).

For preparing the further processing of main capsules containing sub-capsules the solidified subcapsules SKi having different encapsulated functional substances or substance components FSi are preferably suspended in a fluid main capsule matrix phase HKMP in a last substep of the main process step 3 according to this invention. This matrix phase may in turn be either water-based or fat/wax-based. The fluid/solid phase transition temperature of the HKMP is preferably selected according to this invention to be >2 to 5° C. below the phase transition temperature of the SKMPi. The HKMP may in turn contain surfactant layer-forming components GSi according to this invention. These components may form layers at the interfaces between SKMPi and HKMP as well at the interface with the main capsule (HK) and the environment. In the case of water-based HKMP, macromolecular network-forming NMH are also used according to this invention.

Process steps 1-3 are performed for various FSi and subcapsule matrix phases SKMPi coordinated therewith. The resulting suspensions of i subcapsule types in fluid main capsule matrix phase are mixed in defined ratios. These mixing ratios are coordinated for the claimed application of the main capsules with regard to interactions of the FSi and their concentrations that have been optimized for the specific application (e.g., with regard to intensity of physiological effect).

Step 4: MAIN CAPSULE PRODUCTION—In another process step that is preferred according to this invention, the mixed suspension of i subcapsule types in fluid HKMP, produced from steps 1-3 being performed repeatedly wherein the mixed suspension is defined with regard to the quantity ratio and concentration of the various SKi, is sprayed into a cold space and strengthened by solidification or gelation according to the description of mechanisms in step 3, thereby forming the main capsules which are then removed in powder form at the end of step 4.

The resultant main capsules are stored at temperatures below the melting point or glass transition temperature of the capsule matrix phase and layer-forming substance components and substance components mixtures.

A number of optional extra or intermediate steps may be conducted. Both the subcapsules and the main capsules may contain one or more additional layers by spray coating according to this invention.

The fluid phase(s) forming the coatings are sprayed into the cold spray tower below the spraying apparatus installed in the headspace of the tower for the subcapsule suspensions or emulsions (see step 3) or the main capsule suspensions (see step 4) to such an extent that the fluid spray droplets of the coating fluid are deposited on the surfaces of the subcapsules/main capsules that are already partially or completely solidified, these droplets likewise solidifying there as a result of crystallization, vitrification or gelation through withdrawal of heat, forming additional enveloping layers.

An alternative process step 2 can be implemented in the case of fluid FSi. If FSi are to be encapsulated in solution or in fluid dispersion, then according to this invention they are incorporated in the form of disperse droplets into an emulsion having a coordinated fluid subcapsule matrix phase SKMPi as a continuous fluid phase that is not miscible with the droplet fluid phase containing the FSi. In a first process step then instead of wet milling (see solid-based FSi), a fine dispersion of the emulsion is prepared. Depending on the required width of the disperse FSi fluid phase droplet size distribution, the equipment used here according to this invention include rotary/stator dispersion systems or high-pressure homogenizers (broad droplet size distribution) or laminar nozzle dispersion methods such as capillary jet extensional flow dispersion channel (K-JetDSDK) (very narrow droplet size distribution with a standard deviation: s≈≦1%). Cold spraying to solidify the subcapsules is performed subsequently as described in step 3 above. The fluid phase(s) containing the FSi is/are jointly solidified in the cold spray process according to this invention. In principle it is possible to retain them in fluid form but as a rule this is not preferable for stability reasons.

Alternative process steps 3 and 4 can be followed in the case of capsule matrix phases/layers to be solidified with input of heat. Essentially it is also possible according to this invention to replace the cold spray solidification steps for certain substance systems with spray drying steps. In doing so, the corresponding subcapsules/main capsule phases or additional coatings are solidified by thermally-induced crosslinking reactions (e.g., coagulation of protein molecules) or drying (e.g., removal of water from biopolymer solutions or concentrated sugar/salt solutions. Here again, the development of amorphous (vitreous) or crystalline layers can be adjusted as a function of the drying rate according to this invention. The adjustment of drying rate is performed according to this invention preferably via the temperature and the moisture content of the drying air.

There are possibilities for reducing the complexity of the capsule structure and production process. In principle there is the possibility of embedding several FSi in solid or fluid form in one subcapsule matrix phase. The main capsule matrix phase in this case may correspond to an additional coating layer which may optionally also be omitted.

Individual encapsulation and increasing the number of “enveloping layers” (GFi, SKMPi, HKMP) allow an improvement in stability properties of the capsules under storage conditions on the one hand but on the other hand these enveloping layers also allow flexible coordination of different functional components with different properties and sensitivities in a specific manner with the practical boundary conditions.

The purpose of using microcapsules is generally to transport the functional substances/substance components FSi to the preferred site for their release and the most defined possible adjustment of their release kinetics and their on site reactions or reaction kinetics.

An additional purpose of the inventive multimicrocapsules is to release multiple FSi simultaneously or in a defined sequence in immediate proximity to one another and thereby permit a reaction of these released FSi without any significant inferring influences of substance components from the remaining environment. The combined encapsulated FSi are coordinated according to this invention such that synergistic interactions are achieved. For example, the interaction of an antioxidant (FS1) with a compound (FS2) that has a good solubility in an aqueous digestive medium and is of nutritional or pharmacological/medical relevance then leads to oxidation protection for FS2 and thus to improved absorption and bioavailability of FS2.

A plurality of “enveloping layers” around the FSi allows the implementation of specifically adapted protective functions against varying environmental conditions along the path of the multimicrocapsules to their “destination site” (or the site of release of FSi). For example, in the case of transport in the human digestive tract, passage through the following main zones is associated with specific stresses: A. mouth/pharynx (neutral pH; high mechanical stress); B. stomach (low pH; mechanical stress); C. pylorus/small intestine (neutral pH, introduction of enzymes (e.g., fat-soluble lipases (bile)).

In many cases, there are additional increased requirements of mechanical stability and thermal stability prior to absorption in the human gastrointestinal tract (e.g., in preparing foods containing these multimicrocapsules).

For example, nutritional substance components such as water-soluble iron salts (first substance component, FS1) can be transported into the small intestine of volunteers suffering from iron deficiency in such a way that the iron salts are protected from oxidation by means of such multimicrocapsules; then these salts are released from the capsule in synchronization with an antioxidant (second substance component, FS2). The intense interaction of these components in the microenvironment of the capsule protects the iron salt from oxidation and significantly improves the bioavailability of iron, i.e., its absorption in the small intestine.

For the selected example, the enveloping layers allow the adjustment of the thermal, mechanical, pH and enzymatic stability properties. Degradation of the corresponding enveloping layer may also be desirable here to ultimately ensure the release of the FSi at the target site.

In the human digestive tract, fat-based layers, for example, are degraded by the action of lipases and gel layers based on macromolecular networks in water are degraded by enzymes (e.g., cellulases, proteases) in the pylorus or in the small intestine. Adjusting the thickness of such layers makes it possible to shift the site of release of FSi, e.g., along the small intestine, or to influence the FSi release kinetics.

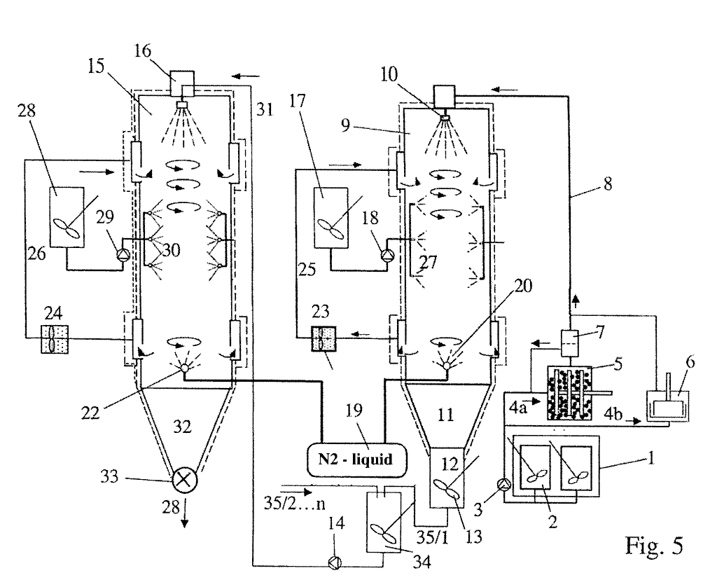

According to the four process steps definitively described above as well as the additional optional/alternative steps, an inventive device for producing multimicrocapsules has been configured. This device is described below and illustrated in detail in the schematic diagram in FIG. 5. The basic apparatus consists of a premixing system, such as a vessel with stirring or mixing elements, for FSi and subcapsule matrix phase(s) SKMPi (1) pumped out of its container (2) by means of a displacement pump (3) either a presuspension (4a) in the case of FSi in solid particle form through a ball mill (5) or in the case of FSi in fluid form a pre-emulsion (4b) pumped through a droplet dispersing device, such as a rotor-stator dispersing system, a high pressure homogenizer, an ultrasound dispersing device, a membrane emulsification device, or a capillary channel microfluidic device (6). By means of a separation device (7) (e.g., filter or sedimentation stage), SFi particles/droplets that are too large in steps 4a/4b are separated and recycled to the milling space of the ball mill/dispersion space of the droplet dispersion system.

The SFi suspensions/emulsions whose particle or droplet size distribution is adjusted are supplied to a high-pressure cold spray system such as a cold spraying tower (9) through a heated pipeline (8). This is where, by means of a spray nozzle (10), the FSi suspension/emulsion is sprayed in fine droplets that solidify by cooling in a cold gas atmosphere (created by spraying and evaporating a fluid gas). In the bottom space (11) of the conically temperature controlled cold spray tower (9), another temperature controlled suspension container (12) with a stirrer (13) is installed according to this invention. The cold sprayed subcapsules are collected in temperature controlled suspension tanks (12) after solidifying and are then suspended in the fluid main capsule matrix phase (HKMP). Surface layers which make it possible to prevent agglomeration/aggregation of the subcapsules (SK) are formed in these by dissolved surfactant substances (GFi).

This subcapsule suspension is sent according to this invention to a second cold spray tower (15) by means of a pump (14) through a pipeline (31) and likewise sprayed through a spray nozzle (16) and solidified in a cold atmosphere.

In both cold spray towers, the cold is preferably achieved according to this invention by injecting nitrogen mist out of a supply tank (19) which contains liquid nitrogen through nozzles (20, 22). To achieve an improved heat transfer from the cold nitrogen mist to the sprayed subcapsules/main capsules and to lengthen their falling time in the cold spray towers (9, 15), a cold gas stream in countercurrent with the direction of spraying is created in the cold spray towers via fans (23, 24) and ascending lines (25, 26). The cold gas is withdrawn in the bottom part of the cold spray towers and the addition to the upper tower areas is accomplished according to this invention preferably tangentially from/to the cylindrical cold spray tower cross sections. This results in spiral flow running helically from bottom to top. The centrifugal forces active in such flows promote separation of the sprayed subcapsules/main capsules on the spray tower walls in the lower conical tower areas (11, 32). The completed multimicrocapsules (main capsules) are usually withdrawn from the second cold spray tower by means of a cellular wheel sluice (33) or an alternative discharge device.

In principle, subcapsules downstream from the first cold spray tower may also constitute a preferred end product and may be withdrawn as such. On the other hand, there is also the possibility of connecting more than two cold spray units in series to implement even more subcapsule categories to be produced with specific devices required for the respective processing of steps 1 to 3.

Several subcapsules produced as described here, present in the form of suspensions of subcapsules in a fluid main capsule matrix phase (HKMP) (35/1 . . . 35/n) at the end of process step 3, i.e., at the outlet of the subcapsule cold spray or spray drying system are preferably mixed in defined ratios coordinated with the administration of the FSi in an intermediate mixing tank 34, and this mixed suspension is sent by pump 14 through a heated pipeline 31 to another cold spray or spray drying unit 15 to perform process step 4 (main capsule production).

To apply additional layers of material to cold sprayed capsules, so-called spray coating nozzles (27, 30) are provided in the inventive cold spray towers. Through these spray nozzles, coating fluids are sprayed from containers (17, 28) by means of pumps (18, 29) onto the capsules falling in the cold spray towers and the coatings are solidified on the surfaces of the capsules due to withdrawal of heat. In principle, each cold spray tower may have one or more such spray coating devices.

The respective spray coating nozzles are then preferably arranged in zones situated one above the other according to this invention in order to produce defined layered coatings.

In the case of the inventive solidification of subcapsules, main capsules or spray layers applied additionally thereto by thermal input of energy and the resulting thermally induced crosslinking reactions or by drying, there is a supply of hot gas/drying gas in the inventive cold spray towers at the site of the cold gas injection (nozzles 20, 22). In this case the nitrogen tank (19) is also replaced by an air heater.

What is claimed is:

1. Multimicrocapsules comprising encapsulated components of at least one nutritionally, pharmacologically, or medically active functional substance, wherein, within an encapsulation comprising one or more layers of a first encapsulating material around a common capsule matrix, a first active functional component in an amount sufficient to provide functional benefits after being ingested by a subject and a component preserving or function enhancing substance present in an amount to preserve or enhance the functional benefits of the active functional component, wherein the encapsulation delays release of the active functional component when the microcapsules are ingested by the subject and provides a synchronous or tuned release of the active functional component and component preserving or function enhancing substance in the intestine of the subject.

2. Microcapsules according to claim 1, wherein the first active functional component is one that is deteriorated by oxidation, the component preserving substance is an antioxidant and the first active functional component is associated with the component preserving substance in the encapsulation.

3. Microcapsules according to claim 1, wherein the first active functional component and component preserving substance are separately encapsulated so that they do not come in contact with each other until after ingestion and release in the intestine of the subject.

4. Microcapsules according to claim 1, which further comprises, within the encapsulation, a second active functional component that is different from the first active functional component in an amount sufficient to provide a different functional benefit or a synergistic functional benefit by interaction with the first active functional component after being ingested by a subject.

5. Microcapsules according to claim 4, wherein the second active functional component is separately encapsulated so that it does not come in contact with the first functional active agent prior to release in the subject.

6. Microcapsules according to claim 1 wherein the common capsule matrix comprises a hydrophobic fat or wax material that suppresses diffusion-based exchange of the active functional component and the component preserving or function enhancing material during storage of the microcapsules prior to ingestion.

7. Microcapsules according to claim 1, wherein the common capsule matrix is a hydrophilic material that forms a gel to separate the first active functional compound from the component preserving or function enhancing material during storage of the microcapsules prior to ingestion.

8. Microcapsules according to claim 1, wherein the first encapsulating material comprises a lecithin compound, a sugar ester, a polyglycerol ester, or a mono- or di-glyceride compound and is present in one or more layers to provide the encapsulation.

9. Microcapsules according to claim 1, wherein the first active functional component is a vitamin, a mineral, a metal compound, an aroma or an oxidizable drug, and the component preserving or function enhancing substance material is an antioxidant, an enzyme, or a halide.

10. Microcapsules according to claim 9, wherein the first active functional compound is an aroma-generating component, and which further comprises a component that is reactable with the aroma generating component to generate and release the aroma when the components contact each other after release from the microcapsules.

11. Microcapsules according to claim 9, wherein the first active functional compound is an aroma-generating component, and which further comprises a component that causes a physiological reaction that sensibilizes human or animal aroma or flavor receptors such that the aroma generating component generates an enhanced aroma impression when released from the microcapsules.

12. A method of providing a nutritionally, pharmacologically, or medically active functional substance to a subject, which comprises administering the microcapsules of claim 1 to a subject in need of such treatment, wherein the microcapsules are administered to provide an amount of the active functional substance to the subject provide functional benefits thereto.

13. The method of claim 11, which further comprises providing the microcapsules in a foodstuff or beverage and the administration of the microcapsules is provided by the consumption of the foodstuff or beverage by the subject.

14. The method of claim 11, specifically for treating a vitamin, mineral or metal deficiency in a subject, by administering to a subject in need of such treatment microcapsules containing as the first active functional component a vitamin, a mineral, or a metal compound.

15. The method of claim 11, specifically for delivering an oxidizable drug to the intestine of the subject, by administering to a subject in need of such treatment microcapsules containing as the first active functional component, an oxidizable drug and an antioxidant as the component preserving substance, such that the first active functional component and antioxidant are preserved by the encapsulation until released in the intestine of the subject.

16. The method of claim 11, specifically for delivering an oxidizable drug to the intestine of the subject, by administering to a subject in need of such treatment microcapsules containing as the first active functional component, an oxidizable drug and an antioxidant as the component preserving substance, such that the antioxidant starts to get released first and prepares the intestinal environment reducing its oxidative capacity and then the first active functional component with delayed release reaches improved bioavailability in the intestine of the subject.

17. A process for producing microcapsules containing at least one nutritionally, pharmacologically, or medically active functional substance which comprises incorporating within an encapsulation comprising one or more layers of a first encapsulating material around a common capsule matrix, a first active functional component in an amount sufficient to provide functional benefits after being ingested by a subject and a component preserving or function enhancing substance present in an amount to preserve or enhance the functional benefits of the active functional component, wherein the encapsulation delays release of the active functional component when the microcapsules are ingested by the subject and provides a synchronous or tuned release of the active functional component and component preserving or function enhancing substance in the intestine of the subject.

18. The process of claim 15, wherein at least two functional substances are provided in the encapsulation, with each substance optionally being encapsulated separately.

19. The process of claim 16, wherein the functional substance components are premixed and are then milled or dispersed in a matrix phase to produce the matrix for a microcapsule or a subcapsule to be placed within the microcapsule.

20. The process of claim 15, wherein the microcapsules or subcapsules are produced by cold spray methods whereby a suspension of the functional solid particles in a fluid matrix phase or an emulsion of the functional fluid droplet phase in a fluid matrix phase are sprayed into a cold gas atmosphere forming fine suspension or emulsion drops and then are solidified in crystalline, gel or vitreous form by withdrawing heat.

21. The process of claim 15, wherein the microcapsules or subcapsules are produced by spray drying methods whereby a suspension of the functional solid particles in a fluid matrix phase or an emulsion of the functional fluid droplet phase in a fluid matrix phase are sprayed into a hot gas atmosphere forming fine suspension or emulsion drops and then are solidified in gel-like or vitreous form by applying heat.

22. The process of claim 15, wherein the encapsulating layers are provided by surfactant substances that are adsorbed from the matrix phases or are spray coated onto the components and common capsule matrix.

23. A device for producing microcapsules containing at least one nutritionally, pharmacologically, or medically active functional substance, comprising static or dynamic mixer or stirrer tools for premixing the functional substance(s) in a fluid matrix phase, a first dispersing device for defined fine dispersion or size reduction of the functional substance(s); a drying device for producing subcapsules and a second dispersing device for producing main capsules which contain multiple subcapsules with the overall device components arranged one after the other in sequential order and are to be operated in this order either batchwise or continuously.

24. The device of claim 23, wherein the first dispersing device comprises a rotor-stator dispersing apparatus, a high pressure homogenizer, an ultrasound dispersing device, a membrane dispersing apparatus, or a microfluidic dispersing channel, the drying device comprises a cold spraying chamber or a spray drying tower, and the second dispersing device comprises a mixing or stirring vessel for sub-capsule suspension mixing or dispersing and a second cold spraying chamber or second spray drying tower.
